(19) United States
(12) Patent Application Publication
(10) Pub. No.: US 2003/0022361 A1
(43) Pub. Date: Jan. 30, 2003

(54) Smart culture vessel

(76) Inventors: Kenneth Houston, Acton, MA (US); Brian T. Cunningham, Lexington, MA (US)

Correspondence Address: Iandiorio & Teska, 260 Bear Hill Road, Waltham, MA, 02451-1018, US

(21) Appl. No.: 10/080,053

(22) Filed: Feb. 21, 2002

Related U.S. Application Data
(60) Provisional application No. 60/270,675, filed on Feb. 22, 2001.

(51) Int. Cl. C12M 1/34; C12Q 1/00

(52) U.S. Cl. 435/287.1; 205/777.5

(57) ABSTRACT

A smart culture vessel for holding a sample to be tested in a culture medium includes a bio-sensor, in the vessel in the culture medium with a sample, having a coating for attracting at least one pathogen expected in the sample and a detection device responsive to the bio-sensor for indicating the presence of the pathogen on the bio-sensor.

### FIELD OF THE INVENTION

This invention relates to a smart culture vessel for providing continuous monitoring and instantaneous detection of a pathogen in a culture medium.

### RELATED APPLICATIONS

This invention claims priority of U.S. Provisional Patent Application Serial No. 60/270,675 filed Feb. 22, 2001.

### BACKGROUND OF THE INVENTION

Often, pathogens cannot be detected directly because their concentration is too low for available tests. Pathogens include various forms of harmful microorganisms, which may be in the form of bacteria, spores, phages, etc. Possible sources include a) body fluids (blood, lymph, urine, spinal, etc.), b) water, c) food and d) article(s) which are suspected of having been contaminated. A culture bottle is a means for increasing the concentration of pathogens by providing an environment which is favorable for growth. For example, in the case of blood cultures, blood is added to a broth containing a base material such as a soybean casein digest and various additives. The culture must be incubated for up to 7 days. At periodic intervals the culture is sampled and tested by manual inspection (for example, evidence of colony growth on agar) or, in the case of automatic inspection systems, the gas in the bottle is sampled for evidence of carbon dioxide in various ways. If carbon dioxide levels indicate growth then a sample is taken and analyzed.

A more recently developed method is based upon polymerase chain reaction (PCR) methods. In this case, a culture must still generally be used. After an appropriate period of growth, the culture is sampled and a sample preparation procedure extracts the DNA of the microorganisms. The DNA is amplified, or copied many-fold, through PCR techniques, and is subsequently detected by use of gel electrophoresis or a DNA chip which deposits the DNA in a pattern that is unique to the target pathogen.

These traditional culturing methods are insensitive and thus take too long to detect the presence of microorganisms, and are not adequately specific. Bottles must be subcultured regularly, increasing the chance of contamination of either the sample or the environment. The procedure is either labor intensive, or in the case of automated systems, requires expensive equipment.

The more recent PCR methods are able to perform testing much more quickly after culturing (perhaps 1 hour). Disadvantages include 1) the issue of various naturally occurring factors inhibiting the PCR replication process, 2) considerable sample preparation is required prior to PCR, including the need for various reagents, and 3) the need for sophisticated and potentially expensive equipment. In addition, considerable culturing (e.g., overnight) is required to achieve a minimum detectable level (on the order of 10⁴ cfu/ml).

### BRIEF SUMMARY OF THE INVENTION

It is therefore an object of this invention to provide an improved, smart, culture vessel.

It is a further object of this invention to provide such an improved, smart, culture vessel which continuously monitors and instantly detects a pathogen in the culture medium.

It is a further object of this invention to provide such an improved, smart, culture vessel which eliminates the need to draw samples for testing, is self contained and reduces the chances of contamination of the culture medium and of the outside environment.

It is a further object of this invention to provide such an improved, smart, culture vessel which is faster at initial detection and subsequent confirmation.

It is a further object of this invention to provide such an improved, smart, culture vessel which does not require sub-culture sampling, sample preparation or use of reagents.

The invention results from the realization that a safe and effective, smart culture vessel which continuously monitors and quickly detects a pathogen in the culture medium can be achieved with a bio-sensor disposed in the culture medium in the vessel, the bio-sensor having a coating which attracts the expected pathogen and a detection circuit for indicating the presence of a pathogen on the bio-sensor.

This invention features a smart culture vessel for holding a sample to be tested in a culture medium including a bio-sensor, in the vessel in the culture medium with a sample, having a coating for attracting at least one pathogen expected in the sample and a detection circuit responsive to the bio-sensor for indicating the presence of a pathogen on the bio-sensor.

In a preferred embodiment the bio-sensor may include an array of bio-sensor elements. Each bio-sensor element may have a different coating for attracting pathogens. The detection circuit may drive the bio-sensor over a range of predetermined frequencies and detect a shift in frequency over time due to an attached pathogen, where the shift in frequency is a shift in the resonant frequency of the bio-sensor. The detection circuit may drive the bio-sensor at a pre-determined frequency, such pre-determined frequency being the resonant frequency of the bio-sensor, and detect a shift in the resonant frequency of the bio-sensor. The detection circuit may continuously drive the bio-sensor and instantaneously detect a shift in frequency. The detection circuit may be external to the vessel.

### BRIEF DESCRIPTION OF THE DRAWINGS

Other objects, features and advantages will occur to those skilled in the art from the following description of a preferred embodiment and the accompanying drawings, in which:

FIG. 1 is a schematic three-dimensional view of a smart culture vessel according to this invention;

FIG. 1A is a schematic diagram of the smart culture vessel system concept in accordance with the present invention;

FIG. 1B is a schematic view of another embodiment of the smart culture vessel in accordance with this invention;

FIG. 1C is a schematic view of the pathogen detection system with a disposable sensor unit;

FIG. 2 is a partial view of a vessel similar to FIG. 1 showing an alternative implementation of the bio-sensor connection;

FIG. 3 is a schematic diagram of a bio-sensor, detection circuit and microprocessor for use in connection with the present invention;

FIG. 4 is a more detailed diagram of one element of the bio-sensor of FIGS. 1 and 2;

FIG. 5 is a schematic flow diagram for an open-loop resonant frequency measurement system for use with the present invention; and

FIG. 6 is a schematic flow diagram for a closed-loop resonant frequency measurement system for use with the present invention.

### DESCRIPTION OF PREFERRED EMBODIMENT

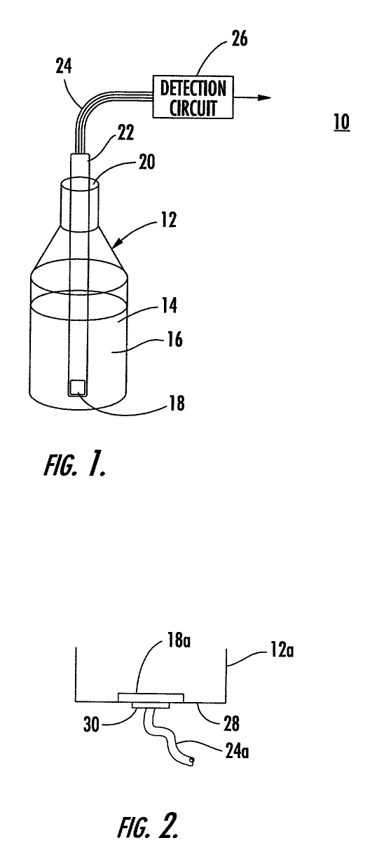

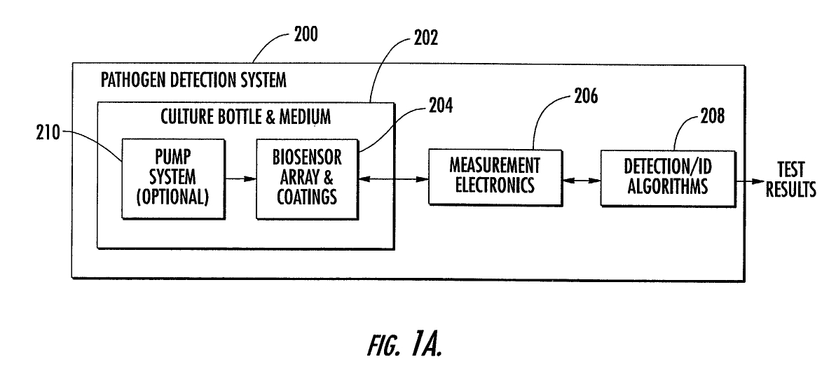

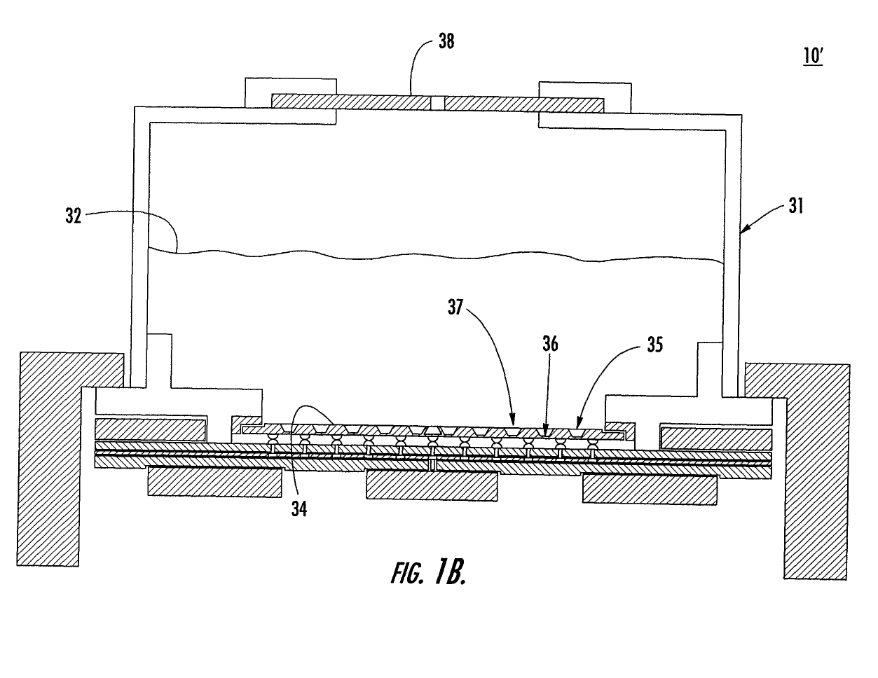

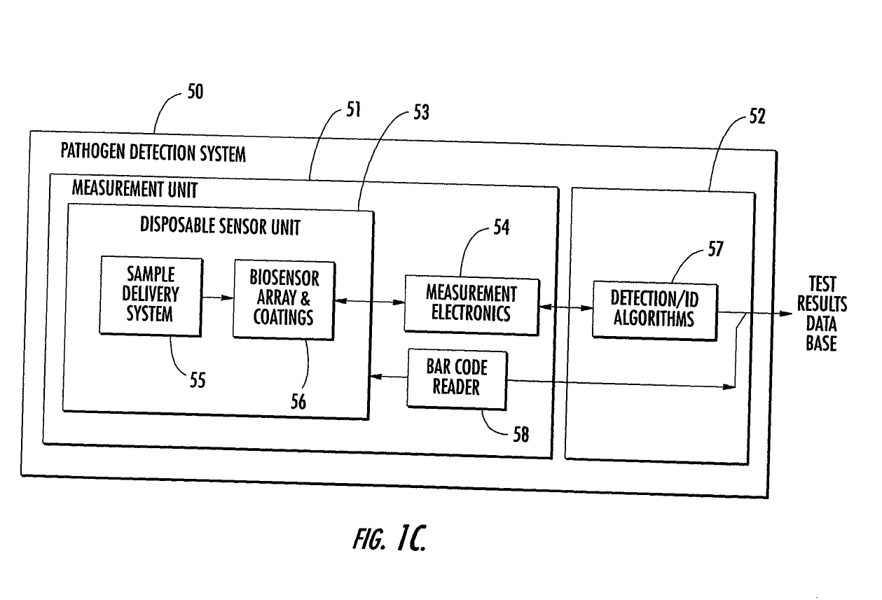

There is shown in FIG. 1 a smart culture vessel 10 according to this invention including a bottle 12 which contains a culture medium 14 in which is a sample 16 to be tested. In accordance with this invention disposed in the bottle is a bio-sensor 18 which is connected electrically through the sealed top 20 of bottle 12 by conduit 22 and electric wires 24 to the detection circuit 26. Alternatively the bio-sensor 18a, FIG. 2, may be mounted on the bottom of bottle 12a with a jack that goes through the bottom 28 of bottle 12a to interconnect with jack 30 at the end of cable 24a.

FIG. 1A shows the smart culture vessel system concept schematically. The pathogen detection system 200 includes the culture bottle and medium 202 including a bio-sensor array and coatings 204. The culture bottle and medium 202 are connected to the measurement electronics 206, and the pathogen detection system 200 also includes detection/identification algorithms 208. A pump system 210 may be included for circulating the culture medium.

There is shown in FIG. 1B a schematic view of another embodiment of the smart culture vessel 10′ according to this invention. The smart culture vessel 10′ includes a vessel 31, which may be a disposable cartridge, containing a culture medium 32 and including a septum 38. Smart culture vessel 10′ also includes a bio-sensor chip 34 containing bio-sensor elements 35, 36, 37 . . . each having a membrane with a coating. Pathogen detection system 50, FIG. 1C, includes a measurement unit 51 and microprocessor 52, measurement unit 51 includes a disposable sensor unit 53 and measurement electronics 54. Disposable sensor unit 53 includes a sample delivery system 55 and biosensor array with coatings 56. Microprocessor 52 includes detection/identification algorithms 57. A bar code reader 58 may also be included in the system. The invention is not limited to any particular type or shape of vessel or container, but may be used with various types of containers or vessels of various shapes, sizes and configurations.

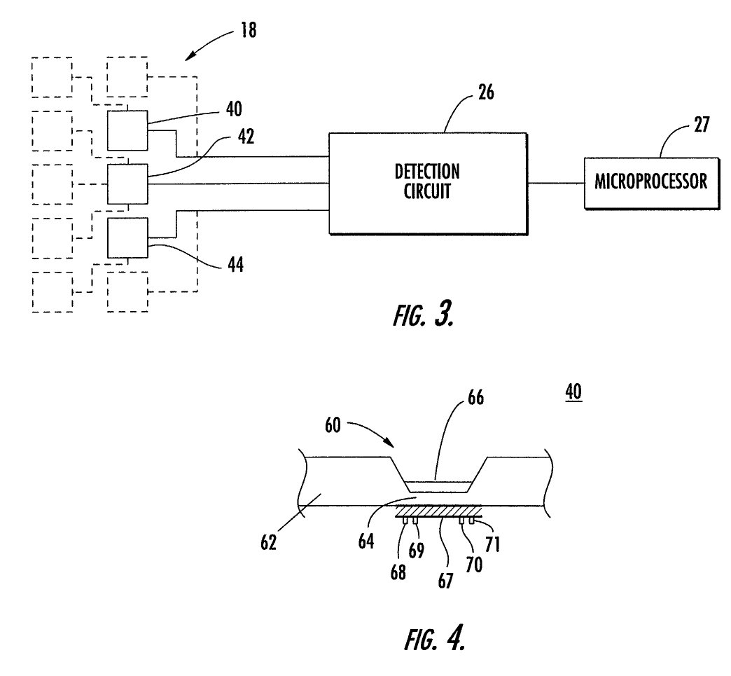

The bio-sensor 18 FIG. 3 may actually include an array of bio-sensor elements 40, 42, 44, . . . which are interconnected to detection circuit 26 and a microprocessor 27. Detection circuit 26 selectively drives each of the bio-sensor elements 40, 42, 44 . . . at a particular frequency, typically the resonant frequency of that bio-sensor in its initial state. Each bio-sensor element is coated with a substance that attracts the particular pathogen of interest. The accumulation of that pathogen on that bio-sensor element due to the attraction to the coating changes the resonant frequency of that particular bio-sensor element which is detected by the detection circuit 26. The presence of the shift of the resonant frequency indicates that the pathogen has arrived and is accumulating on that bio-sensor element. The amount of the shift of the resonant frequency is an indication of the concentration of the detected pathogen. Various analyses may be made using known algorithms. Typical algorithms include the Likelihood Ratio Test (LRT) for simple detection of the presence of a pathogen and the Generalized Likelihood Ratio Test (GLRT) for the combined problem of detection and the estimation of pathogen concentration, which are widely described in the classical detection theory literature. Classical detection theory references are Van Trees, H. L., Detection, Estimation and Modulation Theory, New York, Wiley & Sons, 1968, and Poor, H. Vincent, An Introduction to Signal Detection and Estimation Theory, Springer, 1994.

In this way the sample growing in the culture medium in the bottle is monitored continuously. And instantaneously upon the arrival of the pathogen to the attractive coating a shift in the resonant frequency indicates its presence. This is all done in a closed vessel without need to withdraw samples and run the risk of contamination of the samples by the atmosphere or of the atmosphere by the samples. In addition the detection occurs at the earliest possible moment. There is no reliance on interim indications which trigger an entire sampling and assaying operation.

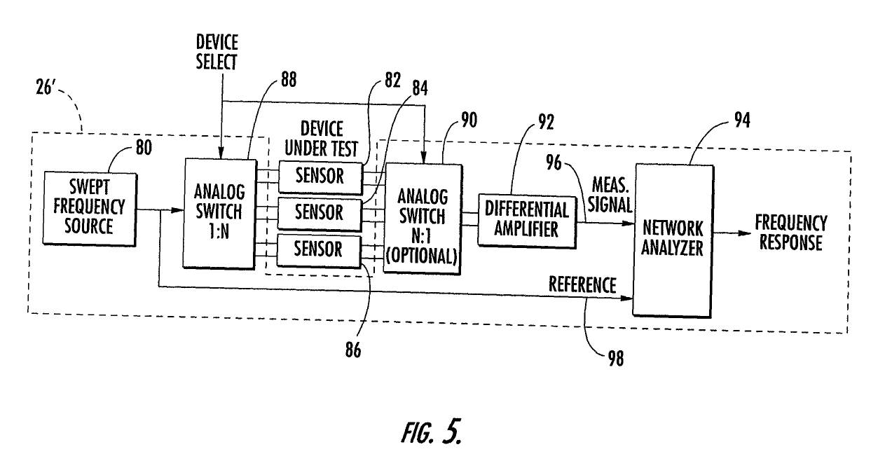

A typical bio-sensor element is exemplified by bio-sensor element 40, FIG. 4, includes a measurement well 60 formed in a silicon chip 62 with a small, thin membrane 64 at the bottom. A coating 66 for attracting pathogens is placed in the bottom of the well against the membrane 64. Below the membrane 64 is typically a piezo-electric coating 67 with two pairs of electrical contacts 68, 69 and 70, 71. One pair of contacts 68, 69 are driven by the detection circuit 26 to vibrate the membrane at its resonant frequency while the other pair of contacts 70, 71 is used to sense the amplitude of membrane motion. The particular coatings used of course depend upon the pathogens that have to be attracted. For example a representative list of pathogens which could be targeted include: candida albicans, escherichia coli, enterococcus species, kelbsiella pneumoniae, pseudomonas aeruginosa, staphyloccus aureus, staphyloccus epiderminis, strepococcus pneumoniae, strepococcus pyogenes, and mycobacterium tuberculosis. The coatings which may be used to attract them are tailored to the targeted pathogens. The coatings may contain affinity ligand reagents that are designed to bind with specific peptides and/or proteins that are present in the pathogen's outer surface.

The description of a typical bio-sensor element may also be found in co-pending U.S. patent application Ser. No. 09/531,970 filed Mar. 20, 2000, and in journal references Brian Cunningham, et al, “Design, Fabrication, and Vapor Characterization of a Microfabricated Flexural Plate Resonator Sensor and Application to Integrated Sensor Arrays”, Sensors and Actuators B, Elsevier Science B. V., 73 (2001) 112-123 and Marc S. Weinberg, et al, “Modeling Flexural Plate Wave Sensors”, IEEE Journal of Microelectromechanical Systems, Vol. 9, No. 3, September 2000, all of which are hereby incorporated herein by reference.

The shift in the resonant frequency indicates the arrival of the pathogen on the attractive coating and this occurs at the instant that the molecules begin to accumulate. By using suitable LRT or GLRT algorithms, a microprocessor 27, FIG. 3, can detect the concentration of the pathogen knowing the particulars of the culture medium.

After the initial decrease in resonant frequency caused by the initial arrival of the pathogen to the attractive coating, the increase of the pathogen on the attractive coating rises exponentially and causes a further, more rapid, decrease in the resonant frequency which confirms the presence of the pathogen on the bio-sensor element.

Circuitry for driving the bio-sensor elements is explained in a co-pending application entitled “SENSOR READOUT CIRCUIT” and filed on Feb. 14, 2002, which is incorporated herein by reference.

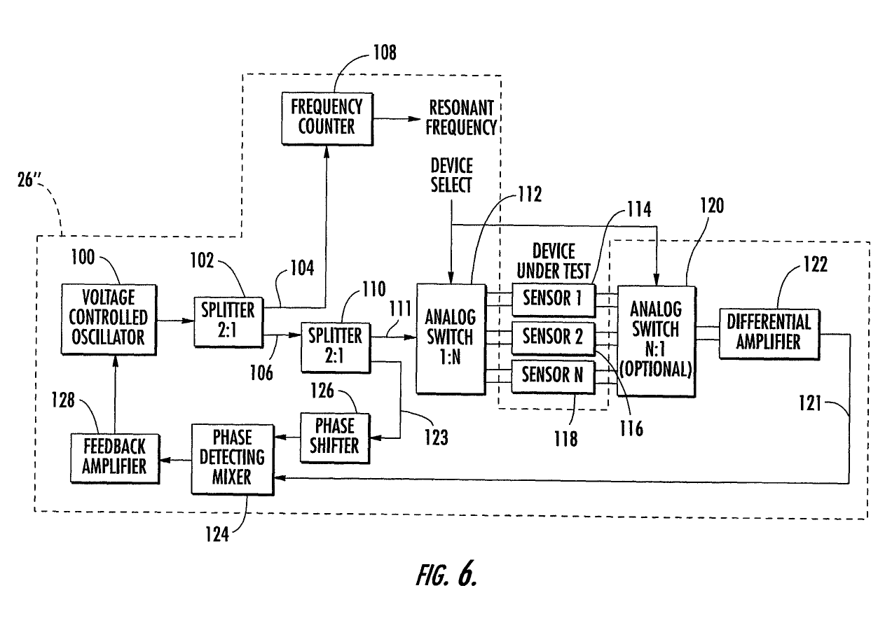

Also, measurement systems to measure resonant frequency and resonant frequency shifts for use with the present invention include open loop and closed-loop measurement systems, FIGS. 5 and 6, respectively.

In FIG. 5, using an open loop approach, a frequency response is obtained, and the resonant frequency is inferred in the processor 27, FIG. 3. In FIG. 5, using a closed loop approach, the system is initialized in the vicinity of the desired resonance, the feedback system drives the voltage controlled oscillator to the resonant frequency, and the frequency shift is measured by microprocessor 27 using a counter or similar device.

In FIG. 5, the detection circuit 26′ includes a swept frequency source 80, typically an oscillator which outputs voltages which are sinusoidal in a series of incremental steps to drive sensors 82, 84 and 86 . . . . Analog switch 88 connects to the sensors 82, 84, 86 . . . and selects which sensor is to be measured, and analog switch 88 runs a series of tests on the selected sensor across a range of frequencies close to the natural frequency of the sensor in order to determine the resonant frequency. Optional analog switch 90 provides more accurate measurement, but detection circuit 26′ may be used without optional analog switch 90 by wiring the electrical contacts 70, 71, see FIG. 4, together in series or parallel. This approach is less costly but provides a correspondingly less accurate measurement. When used, optional analog switch 90 corresponds to the state of analog switch 88. Differential amplifier 92 amplifies the low level voltage output on the selected sensor into a signal which can be measured, that signal representing the motion of the selected sensor. Network analyzer 94 measures both the measured signal 96 and the reference signal 98 and outputs a frequency response indicative of the resonant frequency, which resonant frequency will shift downward over time as pathogens attach to the sensor. Detection circuit 26′ and microprocessor 27, shown in FIG. 3, detect the resonant frequency shift caused by, and indicating the presence of, a pathogen and the concentration of the pathogen knowing the particulars of the culture medium and utilizing typical algorithms as referred to herein.

In FIG. 6, the detection circuit 26″ is such that the voltage controlled oscillator is automatically adjusted to find the resonant frequency and to keep the voltage controlled oscillator at the resonant frequency. Detection circuit 26″ includes a voltage controlled oscillator 100 which outputs a signal to signal splitter 102, which splits the signal into two lower voltage signals 104, 106: signal 104 outputted to frequency counter 108 and signal 106 outputted to a second signal splitter 110. Second signal splitter 110 sends a signal 111 to analog switch 112; analog switch 112 connects to the sensors 114, 116, 118 . . . and selects which sensor is to be measured. Optional analog switch 120 provides more accurate measurement, but detection circuit 26″ may be used without optional analog switch 90 at less cost but with a correspondingly less accurate measurement. When used, optional analog switch 120 corresponds to the state of analog switch 112. Differential amplifier 122 amplifies the low level voltage output on the selected sensor into a signal which can be measured. A signal 121 from the differential amplifier 122 is sent to a phase detecting mixer 124. Signal 123 from second signal splitter 110 is also sent to phase detecting mixer 124 after it passes through phase shifter 126, the latter shifting by 90° the phase of signal 123. Signal 121 represents a measured signal and signal 123 represents a reference signal. The phase detecting mixer 124 causes the voltage controlled oscillator 100 to adjust the frequency to the resonant frequency, with the feedback amplifier 128 acting to amplify and isolate the output of phase detecting mixer 124. By automatically keeping the voltage controlled oscillator 100 at the resonant frequency of the sensor, detection circuit 26″ and microprocessor 27 detect the resonant frequency shift caused by and indicating the presence of a pathogen and the concentration of the pathogen knowing the particulars of the culture medium and typical algorithms as referred to herein, but without sweeping over a range of predetermined frequencies in order to determine the resonant frequency.

The present invention is not limited to any particular measurement system or circuitry and the present invention may be used with established measurement techniques.

Although specific features of the invention are shown in some drawings and not in others, this is for convenience only as each feature may be combined with any or all of the other features in accordance with the invention.

Other embodiments will occur to those skilled in the art and are within the following claims:

What is claimed is:

1. A smart culture vessel for holding a sample to be tested in a culture medium comprising:
a bio-sensor, in the vessel in the culture medium with the sample having a coating for attracting at least one pathogen expected in the sample; and
a detection circuit responsive to the bio-sensor for indicating the presence of a pathogen on the bio-sensor.

2. The smart culture vessel of claim 1 in which the bio-sensor includes an array of bio-sensor elements.

3. The smart culture vessel of claim 2 in which each bio-sensor element has a different coating for attracting pathogens.

4. The smart culture vessel of claim 1 in which the detection circuit drives the bio-sensor over a range of predetermined frequencies and detects a shift in frequency over time due to the attached pathogen.

5. The smart culture vessel of claim 1 in which the detection circuit is external to the vessel.

6. The smart culture vessel of claim 4 in which the range of predetermined frequencies is near the resonant frequency of the bio-sensor.

7. The smart culture vessel of claim 1 in which the detection circuit drives the bio-sensor at a predetermined frequency and detects a shift in frequency due to the attached pathogen.

8. The smart culture vessel of claim 7 in which the predetermined frequency is the resonant frequency of the bio-sensor.

9. The smart culture vessel of claim 6 in which the shift in frequency is a shift in the resonant frequency of the bio-sensor.

10. The smart culture vessel of claim 8 in which the shift in frequency is a shift in the resonant frequency of the bio-sensor.

11. The smart culture vessel of claim 1 in which the detection circuit continuously drives the bio-sensor over a range of predetermined frequencies and detects a shift in frequency over time due to the attached pathogen.

12. The smart culture vessel of claim 1 in which the detection circuit drives the bio-sensor over a range of predetermined frequencies and instantaneously detects a shift in resonant frequency due to the attache d pathogen.

13. The smart culture vessel of claim 1 in which the detection circuit continuously drives the bio-sensor at its resonant frequency and detects a shift in frequency due to the attached pathogen.

14. The smart culture vessel of claim 1 in which the detection circuit drives the bio-sensor at its resonant frequency and instantaneously detects a shift in frequency due to the attached pathogen.
